(19) United States
(12) Patent Application Publication
(10) Pub. No.: US 2006/0247277 A1
(43) Pub. Date: Nov. 2, 2006

(54) Polymorphs of s-omeprazole

(76) Inventors: Yatendra Kumar, Haryana (IN); Mahavir Singh Khanna, Delhi (IN); Mohan Prasad, Haryana (IN)

Correspondence Address: RANBAXY INC., 600 COLLEGE ROAD EAST, SUITE 2100, PRINCETON, NJ, 08540, US

(21) Appl. No.: 10/546,887

(22) PCT Filed: Mar. 1, 2004
(86) PCT No.: PCT/IB04/00535

§ 371 Date: Apr. 28, 2006

(30) Foreign Application Priority Data
Feb. 28, 2003 (IN) 199/DEL/2003

(51) Int. Cl. A61K 31/4439; C07D 403/02

(52) U.S. Cl. 514/338; 546/273.7

(57) ABSTRACT

This invention relates to polymorphic forms of the S-enantiomer of omeprazole which is S-5-methoxy-2-[[(4-methoxy-3,5-dimethyl-2-pyridinyl)-methyl]sulfinyl]-1Hbenzimidazole. The invention also relates to processes for preparing the polymorphic forms. More particularly, it relates to the preparation of two polymorphic forms of Someprazole, referred to as ‘Form I’ and ‘Form 1’ and pharmaceutical compositions that include the ‘Form I’ and ‘Form II’.

### FIELD OF THE INVENTION

The field of the invention relates to polymorphic forms of the S-enantiomer of omeprazole which is S-5-methoxy-2-[[(4-methoxy-3,5-dimethyl-2-pyridinyl)-methyl]sulfinyl]-1H-benzimidazole. The invention also relates to processes for preparing the polymorphic forms. More particularly, it relates to the preparation of two polymorphic forms of S-omeprazole, referred to as ‘Form I’ and ‘Form II’ and pharmaceutical compositions that include the ‘Form I’ and ‘Form II’.

### BACKGROUND OF THE INVENTION

Chemically, omeprazole is 5-methoxy-2-[[(4-methoxy-3,5-dimethyl-2-pyridinyl)-methyl]sulfinyl]-1H-benzimidazole. Omeprazole is a well-known gastric acid secretion inhibitor, and is useful as an anti ulcer agent.

Omeprazole is a racemic mixture of its two single enantiomers, the R-omeprazole and S-omeprazole. U.S. Pat. No. 6,162,816 discloses S-omeprazole in an amorphous form, a partly crystalline form A, and a substantially crystalline form B. PCT patent application WO 02/98423 discloses that S-omeprazole can be isolated as a trihydrate having about 13 to 15% moisture content, although this form has to be stored under refrigerated conditions to provide even limited stability.

### SUMMARY OF THE INVENTION

In one general aspect, there are provided two crystalline polymorphic forms of S-omeprazole, ‘Form I’ and ‘Form II’.

The Form I of S-omeprazole may have the X-ray diffraction pattern of FIG. 1, the infrared spectrum of FIG. 3 and the differential scanning calorimetry curve of FIG. 5.

The Form II of S-omeprazole may have the X-ray diffraction pattern of FIG. 2, the infrared spectrum of FIG. 4 and the differential scanning calorimetry curve of FIG. 6.

In another general aspect there is provided a pharmaceutical composition that includes a therapeutically effective amount of Form I and/or Form II S-omeprazole; and one or more pharmaceutically acceptable carriers, excipients or diluents.

In another general aspect there are provided processes for the preparation of the Form I and Form II S-omeprazole. The processes include preparing a solution of S-omeprazole in one or more solvents; and recovering the S-omeprazole Form I or Form II from the solution thereof by the removal of the solvent.

The solvent may be one or more of lower alkanol, ketone, ester, cyclic ether, nitrile, dipolar aprotic solvent, hydrocarbon, water or mixtures thereof. The lower alkanol may include one or more of primary, secondary and tertiary alcohol having from one to six carbon atoms. The lower alkanol may include one or more of methanol, ethanol, denatured spirit, n-propanol, isopropanol, n-butanol, isobutanol, and t-butanol. In particular, the lower alkanol may include one or more of methanol, ethanol, and denatured spirit.

The ketone may include one or more of acetone, 2-butanone, and 4-methylpentan-2-one.

The ester may include one or more of ethyl acetate, and isopropyl acetate.

The cyclic ether may include one or more of dioxane, and tetrahydrofaran.

The nitrile may include acetonitrile.

The dipolar aprotic solvent may include one or more of dimethylsulfoxide, and dimethylformamide.

The hydrocarbon may include one or more of toluene, and xylene.

The process may include further drying of the product obtained.

Removing the solvent may include one or more of distillation, distillation under vacuum, evaporation, filtration, filtration under vacuum, decantation, and centrifugation.

The Form I or Form II S-omeprazole may be recovered from the solution by filtration. The process may include further forming of the product so obtained into a finished dosage form.

The S-omeprazole Form I or Form II can also be recovered from the solution by adding a suitable non-solvent resulting in the precipitation of the Form I or Form II and removing the solvent there from by filtration, decantation or centrifugation. The non-solvent may be selected from a group of organic solvents in which S-omeprazole Form I and Form II are insoluble or poorly soluble or practically insoluble or partially soluble and is known to a person of ordinary skills in the art.

The process may produce the S-omeprazole Form I having the X-ray diffraction pattern of FIG. 1, the infrared spectrum of FIG. 3 and the differential scanning calorimetry curve of FIG. 5 or Form II having the X-ray diffraction pattern of FIG. 2, the infrared spectrum of FIG. 4 and the differential scanning calorimetry curve of FIG. 6.

The details of one or more embodiments of the inventions are set forth in the description below. Other features, objects and advantages of the inventions will be apparent from the description and claims.

### DESCRIPTION OF THE DRAWINGS

FIG. 1 is a powder X-ray diffraction pattern of S-omeprazole Form I.

FIG. 2 is a powder X-ray diffraction pattern of S-omeprazole Form II.

FIG. 3 is an infrared absorption spectrum of S-omeprazole Form I.

FIG. 4 is an infrared absorption spectrum of S-omeprazole Form II.

FIG. 5 is a differential scanning calorimetry (DSC) curve of S-omeprazole Form I.

FIG. 6 is a differential scanning calorimetry (DSC) curve of S-omeprazole Form II.

FIG. 7 is a microscopic photograph of S-omeprazole Form I.

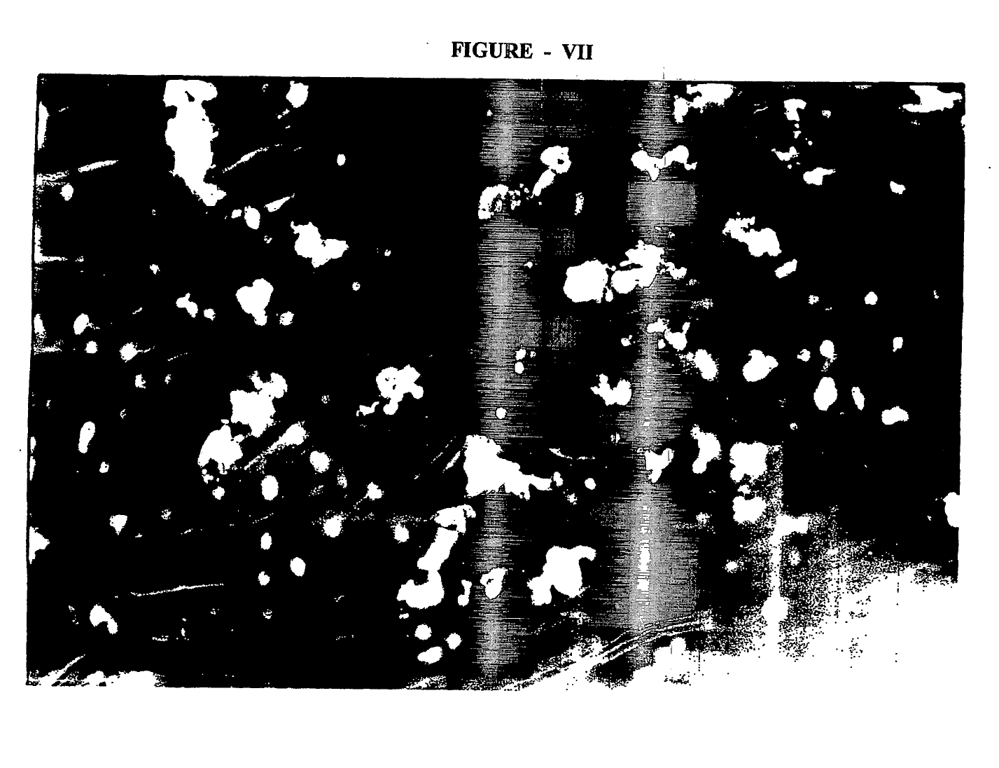

FIG. 8 is a microscopic photograph of S-omeprazole Form II.

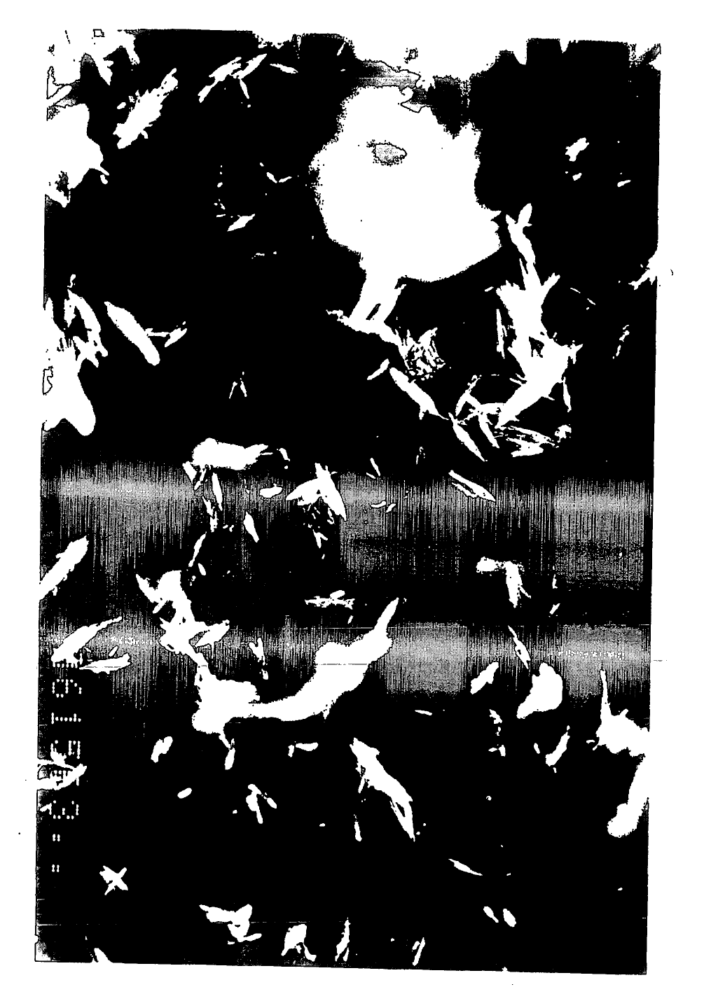

### DETAILED DESCRIPTION OF THE INVENTION

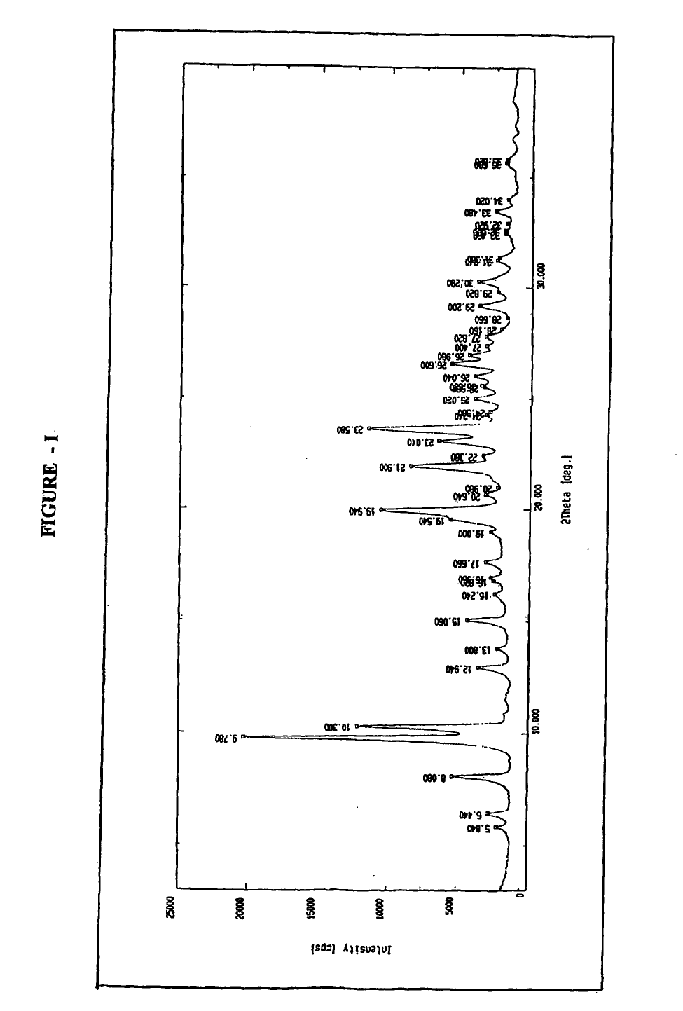

The inventors have found two crystalline polymorphic forms of S-omeprazole, ‘Form I’ and ‘Form II’. The new forms are characterized by their X-ray powder diffraction patterns and infrared spectra as shown in FIGS. 1 and 2, and FIGS. 3 and 4, respectively. The inventors also have developed processes for the preparation of the Form I and Form II, by recovering the Form I and Form II from a solution thereof in a suitable solvent. The inventors also have developed pharmaceutical compositions that contain the Form I and Form II, in admixture with one or more solid or liquid pharmaceutical diluents, carriers, and/or excipients.

The expression ‘S-omeprazole’ refers to the fact that it is substantially free of the R-enantiomer of omeprazole, for example with an enantiomeric excess of 90%, or for example with an enantiomeric excess of 95%. In one aspect, S-omeprazole is in enantiomeric excess of about 99%, or about 99.5%. In another aspect, S-omeprazole is in enantiomeric excess of about 99.8%, or about 99.98%.

In general, Form I S-omeprazole is characterized by a very strong X-ray diffraction peak at about 9.78±0.2. It is further characterized by peaks of strong relative intensifies at about 10.3, 19.9, 21.9, and 23.58±0.2 degrees two-theta; and peaks of medium relative intensities at about 8.08, 12.94, 15.06, 19.54, 23.02, and 26.6±0.2 degrees two-theta. In general, form II of S-omeprazole is characterized by a very strong X-ray diffraction peak at about 10.04±0.2; and peaks of medium relative intensities at about 6.42, 7.44, 8.8, 12.9, 19.44, 20.2, 22.92, 29.5±0.2 degrees two-theta.

In one aspect Form I and Form II S-omeprazole may exist in anhydrous forms as well as hydrated forms. In general, the hydrated forms are equivalent to unhydrated forms and are intended to be encompassed within the scope of the invention. Form I and Form II S-omeprazole contain a water of hydration of at least 7%.

In another aspect Form I and/or Form II S-omeprazole may be sesquihydrates of S-omeprazole.

In general, the solution of S-omeprazole may be obtained by acidifying any salt of S-omeprazole. Alternatively, such a solution may be obtained directly, from a reaction in which S-omeprazole is formed. In another alternative, amorphous form, form A, or form B or any of the various polymorphic forms known in the prior art including solvates, anhydrous or any other polymorphic forms of S-omeprazole may be dissolved in a suitable solvent to obtain a solution.

Any salt of S-omeprazole may be used in the process, including, for example, sodium, potassium, lithium, calcium, magnesium,and tetraalkylammonium salts. The salts of S-omeprazole, or reaction mixture containing S-omeprazole may be prepared using the methods described in U.S. Pat. No. 5,714,504, WO 00/44744; 98/54141; 92/08716; 94/27988, U.S. Pat. Nos. 5,948,789, and 6,124,464 which are incorporated herein as reference. The amorphous form, form A, or form B of S-omeprazole may be obtained using the methods described in U.S. Pat. No. 6,162,816.

Examples of acids, which may be used for acidifying the alkaline salts of S-omeprazole include inorganic acids such as hydrochloric acid, hydrobromic acid, sulfuric acid, phosphoric acid, and nitric acid; or an organic acid such as acetic acid, formic acid, trifluoroacetic acid, methanesulfonic acid, and p-toluenesulfonic acid.

The term “suitable solvent” includes any solvent or solvent mixture in which S-omeprazole has some solubility, including, for example, lower alkanol, ketone, ester, cyclic ether, nitrile, dipolar aprotic solvent, hydrocarbon, water or mixtures thereof. Examples of alkanol include those primary, secondary and tertiary alcohols having from one to six carbon atoms. Suitable lower alkanol solvents include methanol, ethanol, denatured spirit, n-propanol, n-butanol, isopropanol, isobutanol and t-butanol. Examples of ketones include solvents such as acetone, 2-butanone, and 4-methylpentan-2-one. Examples of esters include solvents such as ethyl acetate and isopropyl acetate. Examples of cyclic ethers include solvents such as dioxane and tetrahydrofuran. A suitable nitrile includes acetonitrile. Examples of dipolar aprotic solvents include one or more of dimethylsulfoxide and dimethylformamide. A suitable hydrocarbon solvent includes one or more of toluene and xylene. Mixtures of all of these solvents are also contemplated.

The amount of the solvent used is not limited and will vary depending on such factors as the type of solvent, size of batch and container, temperature of the reaction, and presence or absence of stirring. The crystallization temperature is not limited either, but good results can be obtained by conducting crystallization usually at a temperature of an ice-cold water bath to a room temperature.

In one aspect, additional non-solvent, i.e. a solvent in which S-omeprazole is insoluble or sparingly soluble, can be added to the solution containing S-omeprazole to precipitate the Form I or Form II S-omeprazole before the removal of the solvent and recovering the Form I or Form II S-omeprazole. The precipitation may be spontaneous, depending upon the solvent used and the conditions. For example, precipitation may occur simultaneously on acidification of a solution of an alkaline salt of S-omeprazole. Alternatively, precipitation can be induced by reducing the temperature of the solvent, especially if the initial temperature is elevated. The precipitation may also be facilitated by adding seed crystals of Form I or Form II, or by reducing the volume of the solution.

Generally, the product can be collected by any standard method known in the art such as by filtration, filtration under vacuum, or decantation and drying. Typically, this product will be collected by filtration or centrifugation when any of the solvents within the scope of this process are used. The product obtained may be washed with a suitable solvent and it may be further or additionally dried to achieve the desired moisture values. For example, the product may be further or additionally dried in a tray drier, dried under vacuum and/or in a Fluid Bed Dryer. It may be dried under conditions which avoid degradation of the product, for example air drying below 40° C., or at reduced pressure.

Form I and/or II S-omeprazole so obtained is non sticky and has excellent filtering properties, enabling easy scraping and handling of the filter cake. The Form I and/or II S-omeprazole have good flowability and are thus suitable for formulation into pharmaceutical dosage forms.

The resulting Form I and Form II S-omeprazole may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, and ophthalmic) administration. The oral dosage forms may include solid dosage forms, like powder, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

S-omeprazole is a useful proton pump inhibitor, and thus can be used to treat any condition that would be benefited by administration of a gastric acid secretion inhibitor. In particular, Form I and/or Form II S-omeprazole can be used for prevention and treatment of gastric-acid related conditions in mammals and especially in man, including for example, reflux esophagitis, gastritis, duodenitis, non ulcer dyspepsia, upper gastrointestinal bleeding, stress ulceration, gastrinomas, gastric ulcer, duodenal ulcer, in patients on NSAID therapy, and pre- and postoperatively to prevent aspiration of gastric acid. Further, Form I and/or Form II S-omeprazole may be useful in the treatment of psoriasis as well as in the treatment of Helicobacter infections and diseases related to these.

The present invention is further illustrated by the following examples which are provided merely to be exemplary of the invention and is not intended to limit the scope of the invention. Several variants of these examples would be evident to persons ordinarily skilled in the art.

Methods

X-Ray Powder Diffraction

X-Ray Difractometer, Rigaku Coorperation, RU-H3R

Goniometer CN2155A3

X-Ray tube with Cu target anode

Divergence slits 10, Receiving slit 0.15 mm, Scatter slit 1 0

Power: 40 KV, 100 mA

Scanning speed: 2 deg/min step: 0.02 deg

Wave length: 1.5406 Å

FT-Infrared

Instument:Perkin Elmer, 16 PC

SCAN: 16 scans, 4.0 cm⁻¹

according to the USP 25, general test methods page 1920, infrared absorption spectrum by potassium bromide pellet method.

Differential Scanning Calorimetry

DSC821 e, Mettler Toledo

Sample weight: 3-5 mg

Temperature range: 25-100° C.

Heating rate: 1° C./min

Nitrogen 80.0 mL/min

Number of holes in the crucible: 1

Preparation of Form-I S-omeprazole

### EXAMPLE 1

S-omeprazole potassium (10 g) was added to a mixture of water (60 ml) and toluene (100 ml). The suspension was cooled to 20-25° C. pH of the suspension was adjusted with dilute hydrochloric acid to 7.0-8.5. The reaction mixture was stirred for further 5 min. and the organic layer was separated and then washed with water (40 ml). The organic layer was separated and filtered through a hyflo bed. The hyflo bed was washed with toluene (20 ml). Water (2 ml) was added to it. The solution was cooled to 3-5° C. and stirred further for 1-2 hours. The separated solid was filtered under vacuum and the wet cake washed with chilled toluene (50 ml), followed by n-hexane (100 ml). The product was air dried at 20-25° C. for 15-20 hours. Yield=6.5 g, Moisture Content % (w/w by KF)=8.62%., bulk density=0.23 g/mL.

Powder XRD, IR in KBr and DSC are as shown in FIGS. 1, 3, and 5, respectively.

### EXAMPLE 2

S-omeprazole magnesium (100 g) was added to a mixture of water (600 ml) and ethyl acetate (800 ml). The suspension was cooled to 15-20° C. pH of the suspension was adjusted with dilute hydrochloric acid to 7.0-8.5. The reaction mixture was stirred for further 5 min. and the organic layer was separated. Ethyl acetate (200 ml) was added to the organic layer and then washed with water (200 ml). The organic layer was separated and ethyl acetate recovered under vacuum at 40-45° C. until no more solvent could be removed. The residue was cooled to 25-30° C. and toluene (800 ml) was added to it followed by water (12 ml). The solution was cooled to 3-5° C. and stirred further for 1-2 hours. The separated solid was filtered under vacuum and wet cake was washed with chilled toluene (100 ml), followed by n-hexane (300 ml). The product was air dried at 20-25° C. for 8-10 hours. Yield=85.5 g, Assay on anhydrous basis (by HPLC)=99.97%, Chiral Purity (by HPLC)=99.99%; Moisture Content % (w/w by KF)=16.98%.

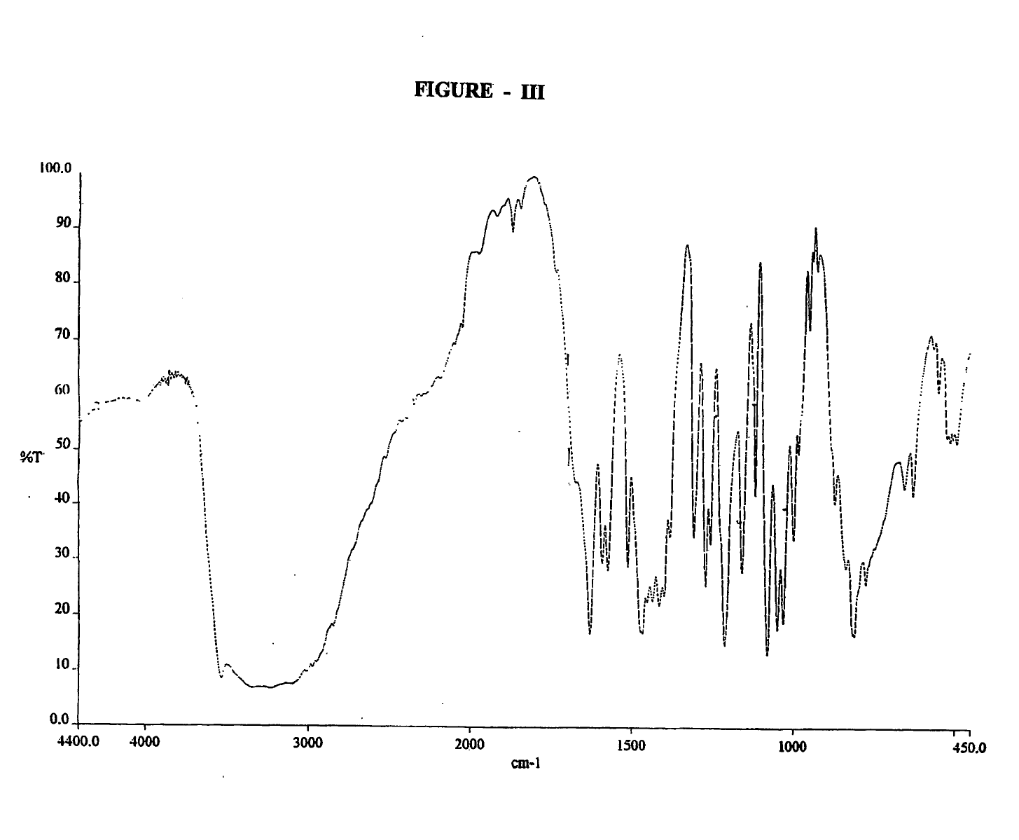

Powder XRD and IR in KBr are similar to those shown in FIGS. 1 and 3, respectively.

Preparation of Form-II S-omeprazole

### EXAMPLE 3

S-omeprazole potasssium (25 g) was added to a mixture of water (150 ml) and ethyl acetate (200 ml). The suspension was cooled to 10-15° C. pH of the suspension was adjusted with dilute hydrochloric acid to 7.0-8.5. The reaction mixture was stirred for further 5 min. and the organic layer was separated. Ethyl acetate (50 ml) was added to the organic layer and then washed with water (100 ml). The organic layer was separated, cooled to 3-5° C. and stirred further for 1-2 hours. The separated solid was filtered under vacuum and wet cake washed with chilled ethyl acetate (50 ml), followed by n-hexane (100 ml). The product was air dried at 20-25° C. for 15-20 hours. Yield=13.2 g; Moisture Content % (w/w by KF)=7.49%, bulk density=0.22 g/mL.

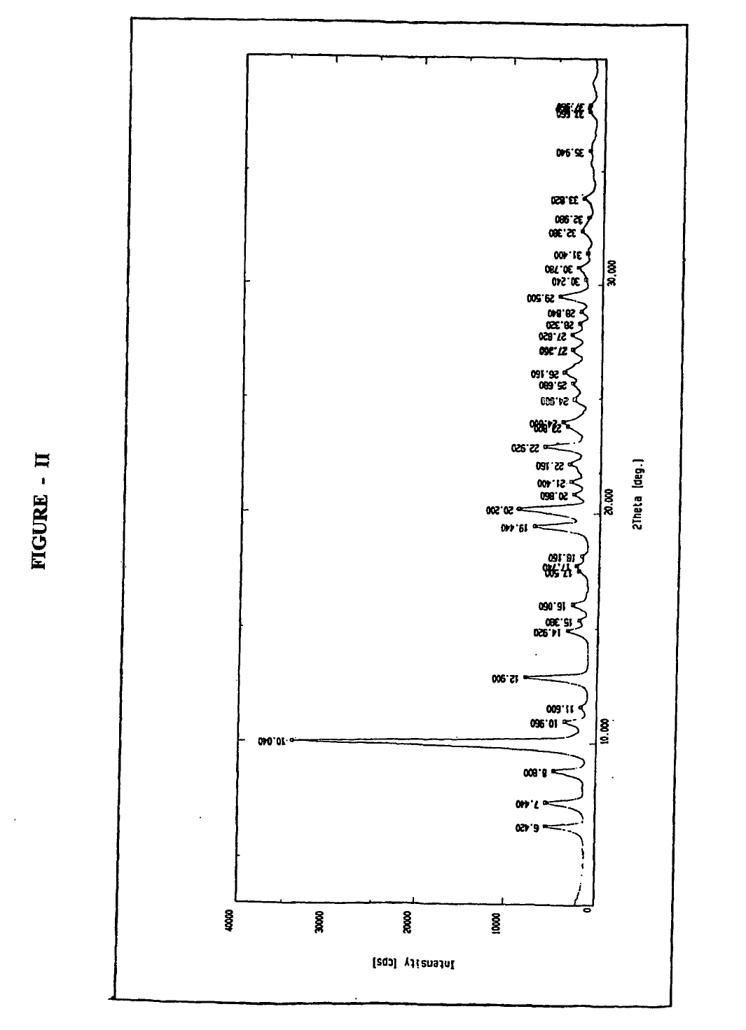

Powder XRD, IR in KBr and DSC are as shown in FIGS. 2, 4, and 6, respectively.

While the present invention has been described in terms of its specific embodiments, certain modifications and equivalents will be apparent to those skilled in the art and are intended to be included within the scope of the present invention.

We claim:

1. Form I S-Omeprazole or hydrates thereof

2. S-omeprazole of claim 1, wherein the S-omeprazole has the X-ray diffraction pattern of FIG. 1.

3. S-omeprazole of claim 1, wherein the S-omeprazole has the infrared spectrum of FIG. 3.

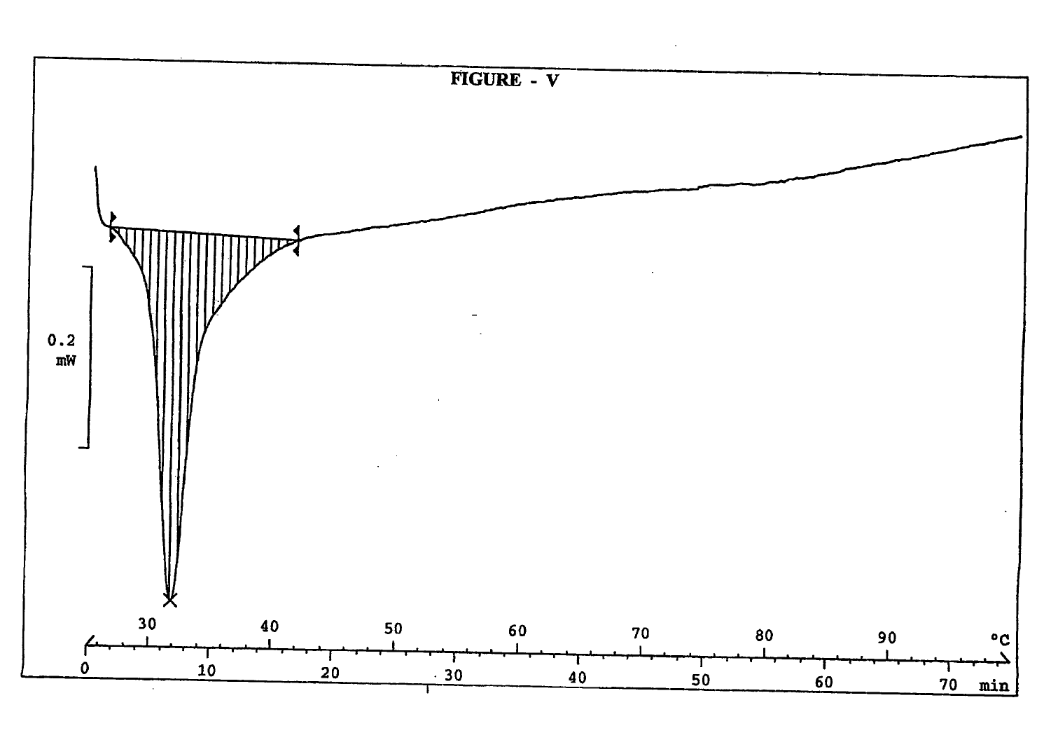

4. S-omeprazole of claim 1, wherein the S-omeprazole has the differential scanning calorimetery curve of FIG. 5.

5. Form I S-omeprazole or hydrates thereof characterized by X-ray diffraction peak at about 9.78±0.2 degrees two-theta.

6. Form I S-omeprazole of claim 5 further characterized by X-ray diffraction peaks at about 10.3, 19.9, 21.9, and 23.58±0.2 degrees two-theta.

7. Form I S-omeprazole of claim 6 further characterized by X-ray diffraction peak at about 8.08, 12.94, 15.06, 19.54, 23.02, and 26.6±0.2 degrees two-theta.

8. A pharmaceutical composition comprising:
a therapeutically effective amount of Form I S-omeprazole or hydrates thereof;
and one or more pharmaceutically acceptable carriers, excipients or diluents.

9. The pharmaceutical composition of claim 8, wherein the S-omeprazole has the X-ray diffraction pattern of FIG. 1.

10. The pharmaceutical composition of claim 8, wherein the S-omeprazole has the infrared spectrum of FIG. 3.

11. The pharmaceutical composition of claim 8, wherein the S-omeprazole has the differential calorimetry curve of FIG. 5.

12. Form II S-omeprazole or hydrates thereof.

13. S-omeprazole of claim 12, wherein the S,-omeprazole has the X-ray diffraction pattern of FIG. 2.

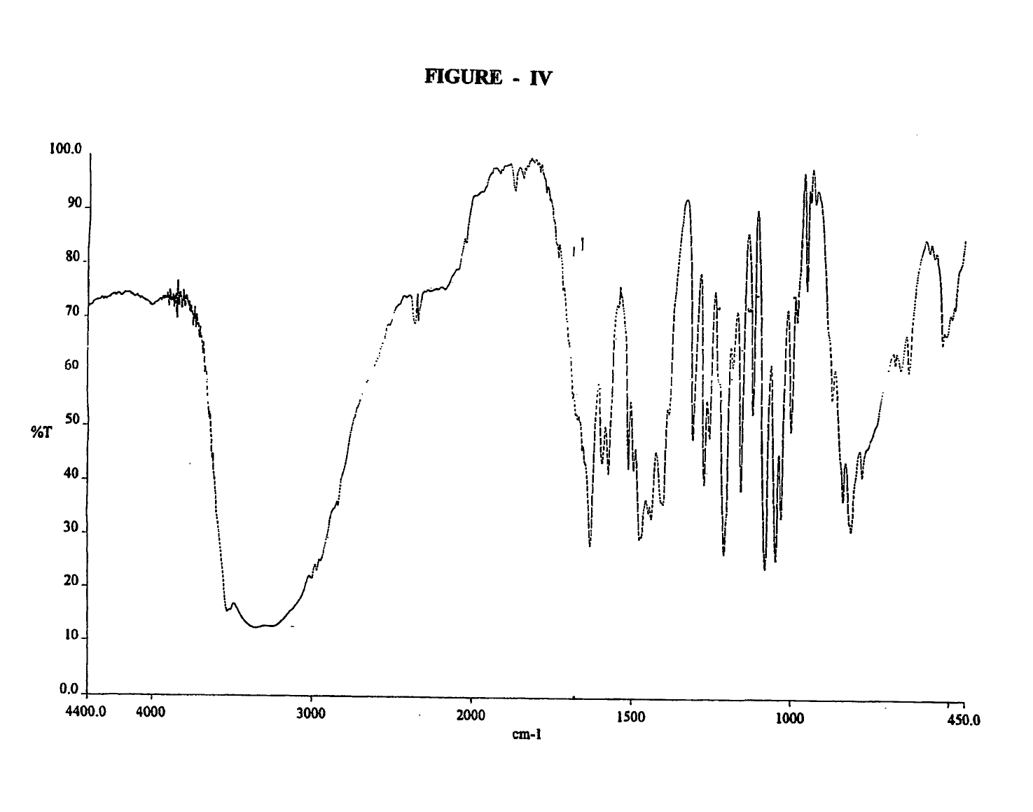

14. S-omeprazole of claim 12, wherein the S-omeprazole has the infrared spectrum of FIG. 4.

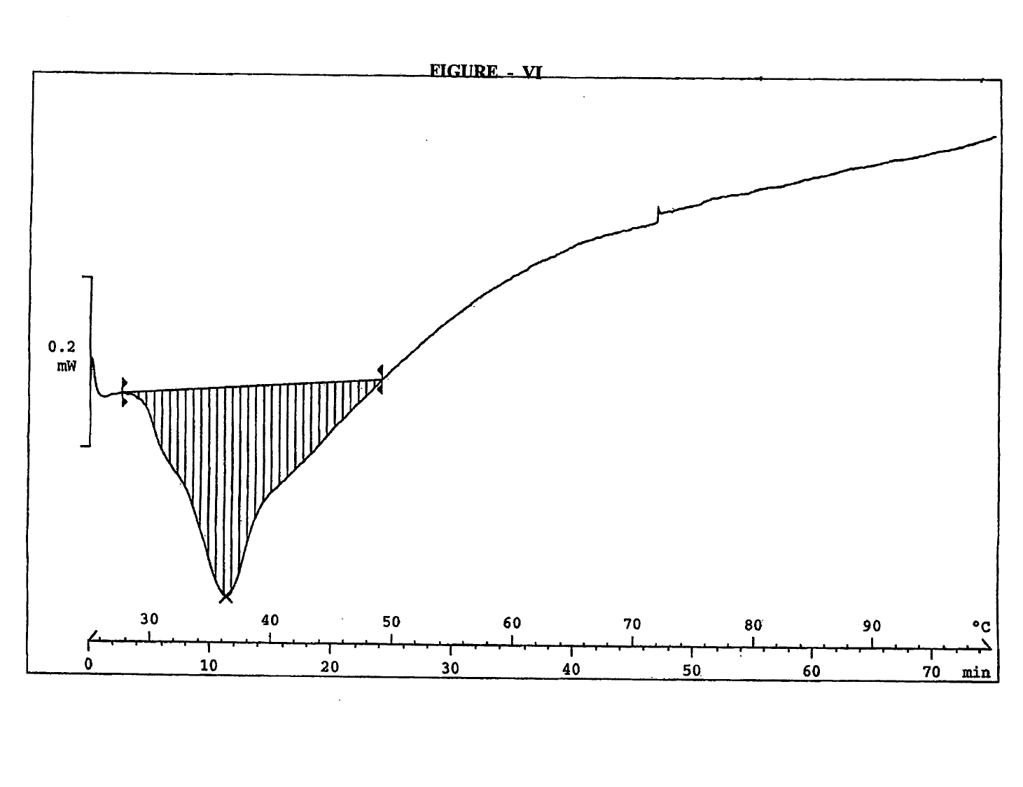

15. S-omeprazole of claim 12, wherein the S-omeprazole has the differential scanning calorimetry curve of FIG. 6.

16. Form II S-omeprazole or hydrates thereof characterized by X-ray diffraction peak at about 10.04±0.2 degrees two-theta.

17. Form II S-omeprazole of claim 16 further characterized by X-ray diffraction peaks at about 6.42, 7.44, 8.8, 12.9, 19.44, 20.2, 22.92, and 29.5±0.2 degrees two-theta.

18. A pharmaceutical composition comprising:
a therapeutically effective amount of Form II S-omeprazole or hydrates thereof;
and one or more pharmaceutically acceptable carriers, excipients or diluents.

19. The pharmaceutical composition of claim 18, wherein the S-omeprazole has the X-ray diffraction pattern of FIG. 2.

20. The pharmaceutical composition of claim 18, wherein the S-omeprazole has the infrared spectrum of FIG. 4.

21. The pharmaceutical composition of claim 18, wherein the S-omeprazole has the differential scanning calorimetry curve of FIG. 6.

22. S-omeprazole of claim 1 or 12 containing water of hydration of at least 7%.

23. S-omeprazole of claim 1 or 12 which is a sesquihydrate.

24. A process for the preparation of the Form I and Form II S-omeprazole or hydrates thereof, the process comprising:
preparing a solution of S-omeprazole in one or more solvents; and
recovering the Form I or Form II S-omeprazole from the solution thereof by the removal of the solvent.

25. The process of claim 24, wherein the solvent comprises one or more of lower alkanol, ketone, , ester, cyclic ether, nitrile, dipolar aprotic solvent, hydrocarbon, water, or mixtures thereof

26. The process of claim 25, wherein the lower alkanol comprises one or more of primary, secondary and tertiary alcohol having from one to six carbon atoms.

27. The process of claim 25, wherein the lower alkanol comprises one or more of methanol, ethanol, denatured spirit, n-propanol, isopropanol, n-butanol, isobutanol, and t-butanol.

28. The process of claim 25, wherein the ketone comprises one or more of acetone, 2-butanone, and 4-methylpentan-2-one.

29. The process of claim 25, wherein the ester comprises one or more of ethyl acetate, and isopropyl acetate.

30. The process of claim 25, wherein the cyclic ether comprises one or more of dioxane, and tetrahydrofuran.

31. The process of claim 25, wherein the nitrile is acetonitrile.

32. The process of claim 25, wherein the dipolar aprotic solvent comprises one or more of dimethylsulfoxide, and dimethylformamide.

33. The process of claim 25, wherein the hydrocarbon comprises one or more of toluene, and xylene.

34. The process of claim 33, wherein the hydrocarbon is toluene.

35. The process of claim 25, wherein removing the solvent comprises one or more of distillation, distillation under vacuum, evaporation, spray drying, freeze drying, filtration, decantation, and centrifugation.

36. The process of claim 25, wherein the Form I or Form II S-omeprazole is recovered from the solution by filtration.

37. The process of claim 25, further comprising additional drying of the product obtained.

38. The process of claim 25, further comprising forming the product obtained into a finished dosage form.

39. A method of treating or preventing a gastric-acid related condition which comprises administering to a patient in need of such treatment a therapeutically effective amount of Form I or Form II S-omeprazole or hydrates thereof.
